(19) United States
(12) Patent Application Publication
(10) Pub. No.: US 2020/0078253 A1
(43) Pub. Date: Mar. 12, 2020

(54) WALKING REHABILITATION ROBOT SYSTEM

(71) Applicant: NATIONAL CHIAO TUNG UNIVERSITY, Hsinchu City (TW)

(72) Inventors: KAI-TAI SONG, NEW TAIPEI CITY (TW); CHUN-LONG KO, TAIPEI CITY (TW)

(21) Appl. No.: 16/176,334

(22) Filed: Oct. 31, 2018

(30) Foreign Application Priority Data
Sep. 7, 2018 (TW) 107131394

(51) Int. Cl. A61H 3/00; G16H 20/30; G16H 50/20

(52) U.S. Cl. CPC A61H 3/00; G16H 20/30; G16H 50/20; A61H 2201/1642; A61H 2201/5064; A61H 2201/165; A61H 2201/1671; A61H 2201/5061; A61H 2201/5071; A61H 2201/5007; A61H 2003/007; A61H 2201/1207

(57) ABSTRACT

A walking rehabilitation robot system is provided, which includes two robot feet, any robot foot of the sound leg will generate a plurality of motion detection signals by first move so that another robot foot learns to move. A control device is electrically connected to each robot foot and receives the motion detection signals transmitted by the robot foot of first move. The control device calculates a first movement track by using the motion detection signals, and then calculates the required motor torque to generate a second movement track according to the first movement track. The control device controls the movement of the other robot foot based on the second movement track. The present invention designs for a user with an inconvenient mobility. The user uses a normal movement of sound half body so that the other robot foot can move the inconveniently moved part immediately.

This application claims priority for Taiwan patent application no. 107131394 filed on Sep. 7, 2018, the content of which is incorporated by reference in its entirety.

### BACKGROUND OF THE INVENTION

### Field of the Invention

The present invention relates to a robot system, particularly to an intelligent robot system applying to walking rehabilitation.

### Description of the Related Art

According to data of WHO, apoplexy has been one of the top three causes of death since 1990 in developed countries. In Taiwan, apoplexy is the first factor causing adult disablement, disabling about seventeen thousands of persons each year. Many patients suffering from apoplexy are paralyzed in one side of their bodies. For example, if a hemorrhage takes place in the left brain hemisphere, the right side of the body is paralyzed, and the patient cannot move the muscle of the right side voluntarily. Such a phenomenon is called a unilateral paralysis. As this type of paralysis is caused by the damage of nerves in the brain, 80% apoplexy patients also suffer from dermotactile insensitiveness or blunt movement.

Further, increasing traffic accidents leads to increasing patients of unilateral disablement. The dynamism of the patients of unilateral disablement is decreased, and the physiological and psychological states thereof are gradually worsened. The patients sitting on wheelchairs are likely to suffer osteoporosis, joint contracture, etc., which will further increase the probability of various diseases and bring about a vicious cycle.

In such a case, the quality of life of the patients and the family members nursing the patients would be affected, and the society and government would spend a vast amount of medical resources and human resources in treating diseases and promoting quality of life for the patients. Therefore, it is a proactive goal to use science and technology to coordinate human bodies and machines to help the disabled persons undertake more physical activities and rehabilitate them.

There have been many prior arts proposing exoskeleton structures to help patients undertake leg movements. However, those prior arts do not consider the walking postures or habitual movements of the user but only pay attention to mechanical operation. Some of those prior arts can be adjusted for different users. However, adjustments for different uses of different statuses will consume much time. Further, those prior arts may fail to detect the intention of the user or fail to learn the progress of rehabilitation. Some of those prior arts can predict the movement of the user. However, the system thereof becomes so bulky that it may impair the movement of the user.

Accordingly, the present invention proposes a walking rehabilitation robot system, which can analyze the movement of the normal side of the user in real time and drive the abnormal side to move with both sides coordinating with each other.

### SUMMARY OF THE INVENTION

The primary objective of the present invention is to provide a walking rehabilitation robot system, which provides a lower-limb exoskeleton robot to assist in the rehabilitation of the persons suffering from unilateral paralysis or movement difficulty (such as that caused by apoplexy), wherein the robot system analyzes normal gaits and provides gait symmetry for the disabled (inconvenient) side of the user, whereby to promote the effect of rehabilitation.

Another objective of the present invention is to provide a walking rehabilitation robot system, which is to be applied to central hospitals or rehabilitation clinics for reducing consumption of medical resources and decreasing occupational injuries of medical personnel, whereby the medical resources can be used more efficiently to promote the effect of rehabilitation.

In order to achieve the abovementioned objectives, the present invention proposes a walking rehabilitation robot system, which is worn by a user, and which comprises two robot feet. Any one of two robot feet generates a plurality of motion detection signals while it is moved. According to the motion detection signals, the other robot foot learns to move. A control device is electrically connected with each of two robot feet. The control device receives the motion detection signals transmitted by the robot foot moving firstly and works out a first motion track according to the motion detection signals. The control device further uses the first motion track and the motion detection signals to work out the torques the motors of the other robot feet should output, whereby the motors of the other robot foot output corresponding torques to drive the other robot foot to generate a second motion track symmetric to the first motion track.

In the present invention, each robot foot includes a first link device, a first rotation device, a second slink device, a second rotation device, a bottom sustaining device. The first link device is electrically connected with the control device. While moving, the first link device transmits a first force signal to the control device. The first rotation device is disposed at one end of the first link device and electrically connected with the control device. While the action of the user rotates the first rotation device, the first rotation device generates a first rotation signal and transmits the first rotation signal to the control device. Alternatively, the control devices controls the first rotation device to rotate; while rotating, the first rotation device drives the first link device to move. The second rotation device is disposed at the other end of the first link device and electrically connected with the control device. While the action of the user rotates the second rotation device, the second rotation device generates a second rotation signal and transmits the second rotation signal to the control device. Alternatively, the control devices controls the second rotation device to rotate. The second rotation device is disposed at one end of the second link device. The second link device is electrically connected with the control device. While the second rotation drives the second link device to move, the second link device generates a second force signal and transmits the second force signal to the control device. The bottom sustaining device is disposed at the other end of the second link device and electrically connected with the control device. The bottom sustaining device sustains the sole of the user. While the user moves one of his soles, the corresponding bottom sustaining device transmits pressure sensation signals to the control device, whereby the control device can learn the movement of the bottom sustaining device. The control device works out a first motion track according to the variations of the first force signal, the second force signal, the first rotation signal, the second rotation signal, and the pressure sensation signals.

In the present invention, the first link device includes a first force sensor, which detects the first force signal generated in the movement of the first link device according to the variation of resistance. The second link device includes a second force sensor, which detects the second force signal generated in the movement of the second link device according to the variation of resistance.

In the present invention, the bottom sustaining device includes a plurality of pressure sensors. The pressure sensors detect the applied forces at different positions of the sole of the user to acquire the pressures at different positions. According to the variations of pressures, the control device learns the movement of the bottom sustaining device and works out the center of the pressures on the sole.

In the present invention, the control device includes a computer, motor drivers, microcontrollers, and power supplies.

The present invention further comprises a waistband assembly. The waistband assembly is annularly disposed around the waist of the user, carrying the control device and coupling two robot feet.

In the present invention, each of two robot feet includes a gravity compensator. The gravity compensator is electrically connected with the control device and able to compensate the robot foot for the gravitational influence, which appears while the robot foot is moving.

In the present invention, the first motion track is used to work out the torques the motors should output for generating the second motion track in the technologies of Inverse Reinforcement Learning (IRL) and Q-learning.

In the present invention, the control device uses the IRL method to analyze the translation, rotation, and acting force in the first motion track and uses the Q-learning method to acquire the optimized input actions corresponding to the translation, rotation, and acting force, whereby the control device can work out the torques the motors should output for generating the second motion track.

Below, embodiments are described in detail in cooperation with the attached drawings to make easily understood the objectives, technical contents, characteristics and accomplishments of the present invention.

### BRIEF DESCRIPTION OF THE DRAWINGS

FIG. 1 is a perspective view schematically showing a walking rehabilitation robot system according to one embodiment of the present invention;

FIG. 2 is a block diagram schematically showing the architecture of a walking rehabilitation robot system according to one embodiment of the present invention;

FIG. 3 is a diagram schematically showing a bottom sustaining device and pressure sensors of a walking rehabilitation robot system according to one embodiment of the present invention;

FIG. 4 is a diagram schematically showing the operation of a walking rehabilitation robot system according to one embodiment of the present invention;

FIG. 5 is a diagram schematically showing the signals generated by a first rotation device while the user moves his hip joint according to one embodiment of the present invention;

FIG. 6 is a diagram schematically showing the signals generated by a second rotation device while the user moves his knee joint according to one embodiment of the present invention;

FIGS. 7a-7f are diagrams schematically showing the signals generated by a bottom sustaining device while the user moves his ankle joint according to one embodiment of the present invention.

### DETAILED DESCRIPTION OF THE INVENTION

For a user who is uncomfortable unilaterally or suffers unilateral paralysis, his/her personal motivation is closely related with the effect of rehabilitation. The present invention calculates the gait of the normal left or right half body and uses the result to control the disabled half body, making both feet have symmetric and coordinate gaits and promoting the effect of rehabilitation.

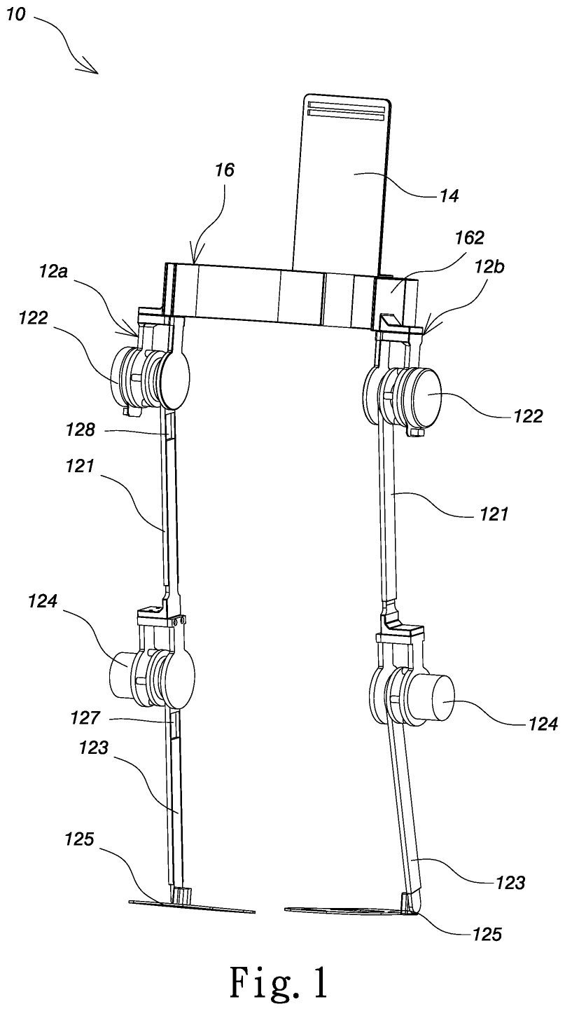

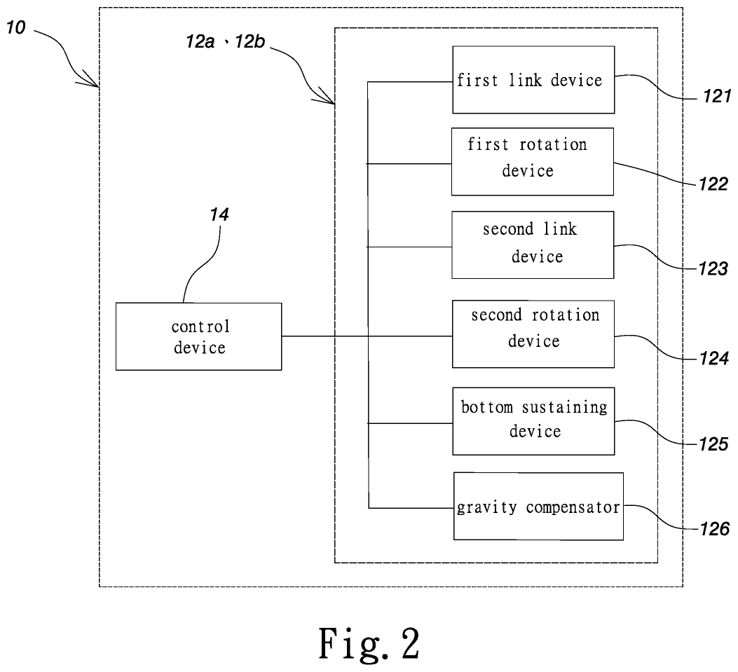

Refer to FIG. 1 and FIG. 2. The present invention proposes a walking rehabilitation robot system 10, which comprises two robot feet 12a and 12b and a control device 14. The control device 14 is electrically connected with two robot feet 12a and 12b. In one embodiment, the control device 14 includes a computer, motor drivers, microcontrollers, and power supplies. In one embodiment, the present invention further comprises a waistband assembly 16. The waistband 16 is annularly disposed around the waist of the user, carrying the control device 14 and coupling two robot feet 12a and 12b, whereby the control device 14 can be disposed on the back of the user.

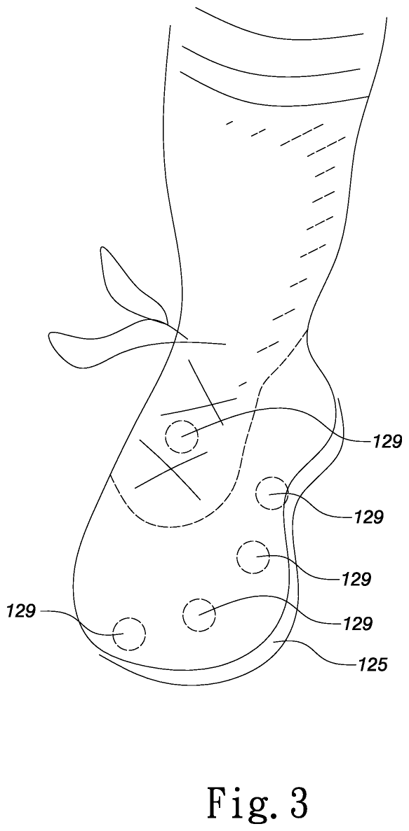

Each of robot feet 12a and 12b includes a first link device 121, a first rotation device 122, a second link device 123, a second rotation device 124, a bottom sustaining device 125, and a gravity compensator 126. In one embodiment, each of the first and second rotation devices 122 and 124 is a rotation motor. The control device 14 is electrically connected with the first link device 121, the first rotation device 122, the second link device 123, the second rotation device 124, the bottom sustaining device 125, and the gravity compensator 126. The first rotation device 122 is disposed at one end of the first link device 121, and the second rotation device 124 is disposed at the other end of the first link device 121. The second rotation device 124 is coupled to one end of the second link device 123. The other end of the second link device 123 is coupled to the bottom sustaining device 125. Refer to FIG. 3 also. The bottom sustaining device 125 includes a plurality of pressure sensors 129. The present invention does not limit the appearance of the bottom sustaining device 125 as long as the bottom of the bottom sustaining device 125 has a plurality of pressure sensors 129. In one embodiment, the bottom sustaining device 125 is in form of a shoe. The present invention does not limit the position where the gravity compensator 126 is located as long as the gravity compensator 126 is electrically connected with the control device 14.

Next is described the operation of the walking rehabilitation robot system of the present invention. Refer to FIG. 1 and FIG. 2 again. In one embodiment, the waist assembly 16 includes a stretchable element 162, which enables the waistband assembly 16 to be adjusted according to the waist girth of the user. Each of the first link device 121 and the second link device 123 is telescopic, whereby the first rotation device 122 can be modified to the altitude of the hip joint of the user and the second rotation device 124 can be modified to the altitude of the knee of the user. In order to make the user wear the system conveniently, fixing elements, such as girds or hook-and-loop straps (not shown in the drawings), are disposed on the first link device 121 and the second link device 123, whereby the user can fix the first link device 121 and the second link device 123 to the thighs and legs.

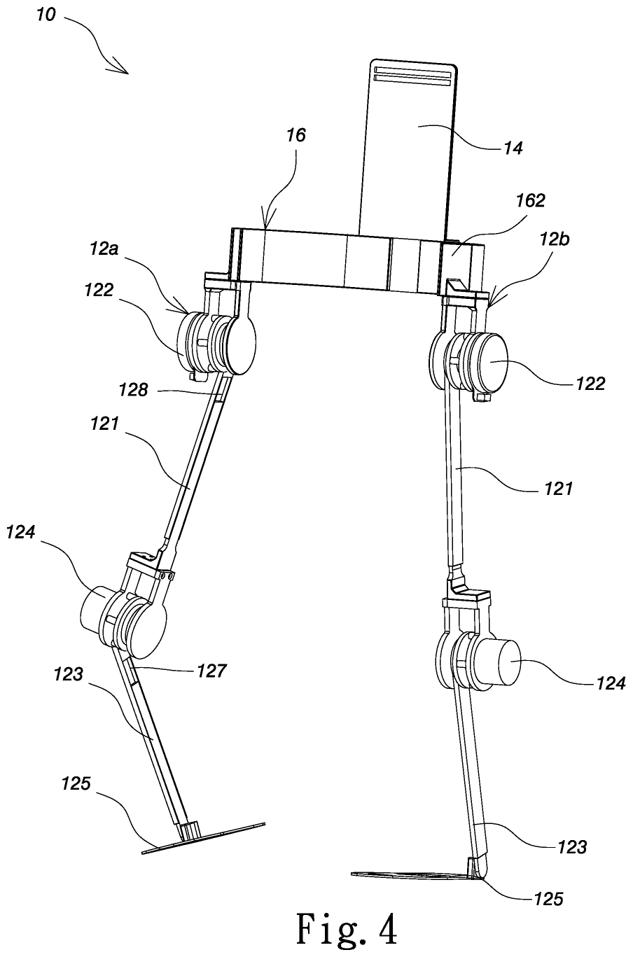

Refer to FIG. 2 again, and refer to FIG. 4. While a user suffering unilateral malfunction wears the walking rehabilitation robot system 10 of the present invention, the user uses his normal foot to drive the robot foot 12a to move. Thereby, the robot foot 12a moves firstly and generates a plurality of motion detection signals. In detail, while the user raises his normal foot, his sole will gradually leave the bottom sustaining device 125 and the pressure sensors 129 thereon. At this instant, the pressure sensors 129 detect the variations of the pressures the sole applies to the pressure sensors 129. The operation of the pressure sensors 129 is based on the relationship between the partial pressure and the resistance value. The greater the pressure, the larger the output voltage. The present invention may use Equation (1) to acquire the pressure variation at the pressure center.

$$CoP = \frac{{\sum }_{i = 1}^{N} {m}_{i} {x}_{i}}{M} \qquad ( 1 )$$

wherein CoP is the Center of Pressure of the pressure sensors 129; N is the number of the pressure sensors 129; in is the voltage value acquired by each pressure sensor 129; x is the distance from the pressure sensor 129 to the end point of the bottom sustaining device 125 (the heel of the user). The pressure sensors 129 transmit the values of the abovementioned parameters to the control device 14, and the control device 14 undertakes computation to acquire the center of pressure according to equation (1). While the user raises his sole, his knee will gradually curve and drive the second rotation device 124 to rotate. The second rotation device 124 generates a second rotation signal and transmits the second rotation signal to the control device 14 through the NI CAN BUS. The second rotation signal includes the angular speed and current of the motor. At this time, the second link device 123 is driven to move by the second rotation device 124 and generates a second force signal. In one embodiment, a second force sensor 127 is disposed on the second link device 123 and below the second rotation device 124, whereby to form a Wheatstone bridge. The independent resistor of the second force sensor 127 is about 350 ohm. However, the present invention does not limit that the independent resistor of the second force sensor 127 must be about 350 ohm. The movement of the second link device 123 makes the resistance of the second force sensor 127 vary. For example, the resistance becomes smaller with increasing force. Thereby is obtained the second force signal. The second link device 123 then transmits the second force signal to the control device 14. While the user moves, the hip joint also moves in addition to the lift-up of the sole and the rotation of the knee joint so that the thigh can move. At this time, the first rotation device 122 rotates according to the actions of the user. The first rotation device 122 exchanges information with the control device 14 through the NI CAN BUS, generating a first rotation signal and transmits the first rotation signal to the control device 14. The rotating first rotation device 122 drives the first link device 121 to move. The first link device 121 may include a first force sensor 128, which is disposed on the first link device 121 and below the first rotation device 122. The operation principle of the first force sensor 128 is the same as that of the second force sensor 127. The movement of the first link device 121 makes the resistance of the first force sensor 128 vary. Thereby is obtained a first force signal. The first shaft device 121 then transmits the first force signal to the control device 14. While the user uses the lower limb of the normal left or right half body to move the robot foot 12a, the gravity compensator 126 will compensate the user for the gravity generated by the movement of the robot foot 12a, whereby the user will not sense the weight of the robot foot 12a while he is moving the lower limb of the normal left or right half body, and whereby the motion detection signals detected in the movement of the user are the same as those generated in normal walking movement. The present invention does not particularly demand that a particular one of the robot feet 12a and 12b should be the robot foot moving firstly. Which one of the robot feet 12a and 12b is to walk firstly is dependent on which one of the left and right half bodies is the normal half body. No matter which one of the robot feet 12a and 12b is moved, the control device 14 can always learn the gait of the user and control the other robot 12a or 12b to move.

While the user makes a single step from a static sate to another static state, the control device 14 can learn the variations of the motion detection signals, such as the pressures signals, the first rotation signal, the first force signal, the second rotation signal, the second force signal, etc. and work out the first motion track. Equations (2) and (3) are respectively the dynamic-state equations of the hip joint and the knee joint of the movable left or right half body of the user. Equations (2) and (3) are respectively expressed by

Tₕ=[(m₁+m₂)L₂²m₂L₃²+2m₂L₂L₃ cos(θ₃)]\ddot{θ}₂+[m₂L₃²+m₂L₂L₃ cos(θ₃)]\ddot{θ}₃−2m₂L₂L₃ sin(θ₃) \dot{θ}₂\dot{θ}₃−m₂L₂L₃ sin(θ₃) \dot{θ}₃²+(m₁+m₂)gL₂ sin (θ₂)+m₂gL₃ sin(θ₂+θ₃) (2)

Tₖ=[m₂L₃²+m₂L₂L₃ cos(θ₃)]\ddot{θ}₂+m₂L₃²\ddot{θ}₃+m₂L₂L₃ sin(θ₃) \dot{θ}₂²+m₂gL₃ sin(θ₂+θ₃) (3)

wherein m₁ and m₂ are respectively the masses of the terminals of the thigh and leg of the user; L₂ and L₃ are respectively the lengths of the thigh and leg of the user; g is the gravitational acceleration; θ₂ and θ₃ are respectively the angular coordinates of the rotations of the hip joint and the knee joint. Then, Equation (4), which involves different variables (Xi), such as angle, angular speed and angular acceleration, is added to Equation (3) to obtain Equation (5). Equation (5) may be further expressed by variable terms to obtain Equation (6). Equations (4), (5) and (6) are respectively expressed by

Tₑₓₜ+Tₘ=Dᵢᵢ\ddot{θ}ᵢ+Dᵢⱼ\ddot{θ}ⱼ+Dᵢⱼⱼ\dot{θ}ⱼ²+Dᵢⱼₖ\dot{θ}ⱼ\dot{θ}ₖ+Dᵢ+D_{D}\dot{θ}+f (4)

Tₕ_{ₘ}=[(m₁+m₂)L₂²+m₂L₃²]\ddot{θ}₂+2m₂L₂L₃ cos(θ₃) \ddot{θ}₂m₂L₃²\ddot{θ}₃+m₂L₂L₃ cos(θ₃) \ddot{θ}₃2m₂L₂L₃ sin(θ₃) \dot{θ}₂\dot{θ}₃−m₂L₂L₃ sin(θ₃) \dot{θ}₃²+(m₁+m₂)gL₂ sin (θ₂)+m₂gL₃ sin(θ₂+θ₃)+D_{D}\dot{θ}₂+f (5)

\hat{X}ₕ=[1 \ddot{θ}₂ cos(θ₃) \ddot{θ}₂ \ddot{θ}₃ cos(θ₃) \ddot{θ}₃−sin(θ₃) \dot{θ}₂\dot{θ}₃−sin(θ₃) \dot{θ}₃² sin(θ₂) sin(θ₂+θ₃) \dot{θ}₂ Sign(−\dot{θ}₂)] (6)

wherein Tₑₓₜ is the external torque applied by the user; Tₘ is the rotation torque of the first or second rotation device; D_{D} is the damping coefficient; f is the friction torque of the first rotation device 122 or the second rotation device 144 during rotation. In the present invention, it is expected that the exoskeleton can compensate for the gravitational force before the robot system provides auxiliary torque. Therefore, the control device does not consider the external force applied by the user but only considers the rotation torque applied by the motors at the beginning in calculating gravitational compensation. Thus, let the external torque Tₑₓₜ applied by the user be zero. The control device 14 can work out the motor torques of the other robot foot according to the first motion track and the motion detection signals. For example, if the robot foot 12a moves, the control device 14 calculates the motor torques of the other robot foot 12b and controls the other robot foot 12b to generate a second motion track symmetric to the first motion track. According to the second motion track, the control device 14 outputs the corresponding rotation angles and applied forces to control the first rotation device 122 of the robot foot 12b to rotate and drive the first link device 121 to move and controls the second rotation device 124 to rotate and drive the second link device 123 to move. The movement of the robot foot 12b drives the lower limb of the paralyzed left or right half body to move in coordination with the movement of the lower limb of the normal half body.

After the control device receives the motion detection signals, such as the variations of the sole pressure signals, the first rotation signal, the first force signal, the second rotation signal, the second force signal, etc., the control device calculates the first motion track, i.e. works out the walking mode of the lower limb of the normal half body of the user. Then, the control device calculates the torques of the motors of the other robot foot and controls the other robot foot to generate the second motion track symmetric to the first motion track. The control device uses the Inverse Reinforcement Learning (IRL) method and the Q-learning method to generate the second motion track according to the first motion track and the motion detection signals. For example, the control device uses the IRL method to analyze the translation, rotation, etc. to learn the applied forces and then uses the Q-learning method to acquire the optimized inputs corresponding to the translation, rotation and applied forces. Thereby, the control device can work out the motor torques required by the second motion track.

The algorithm of the present invention mainly includes the IRL method and the Q-learning method, which are respectively used to learn the habit of the user and executes the auxiliary torque of the expected decision. The learned behaviors enable the disabled half body to realize expected walking movements. In a broad sense, the IRL method is to learn the specialist demonstration preference to describe the observed behaviors. The forces and postures applied by the leg of the healthy half body are used as inputs. The actions executed between states, such as the motions after force is applied, are defined in advance. The acquired data are divided into several states. The behaviors expressed by the reward functions are acquired through the gait track demonstration. In different states, the next action is determined according to the reward value, whereby the leg of the paralyzed half body of the user can obtain appropriate torques. The torques are transformed into the currents input to motors, whereby the robot foot, which the paralyzed leg relies on, is controlled to move.

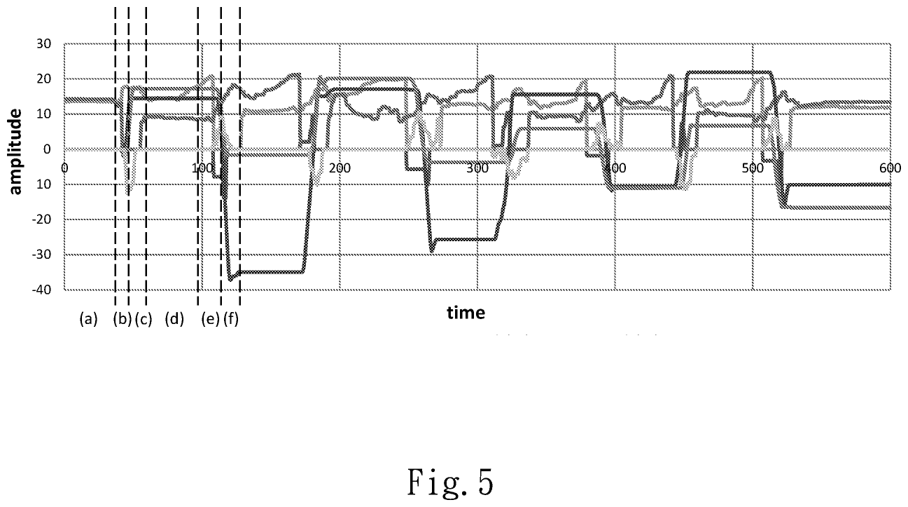

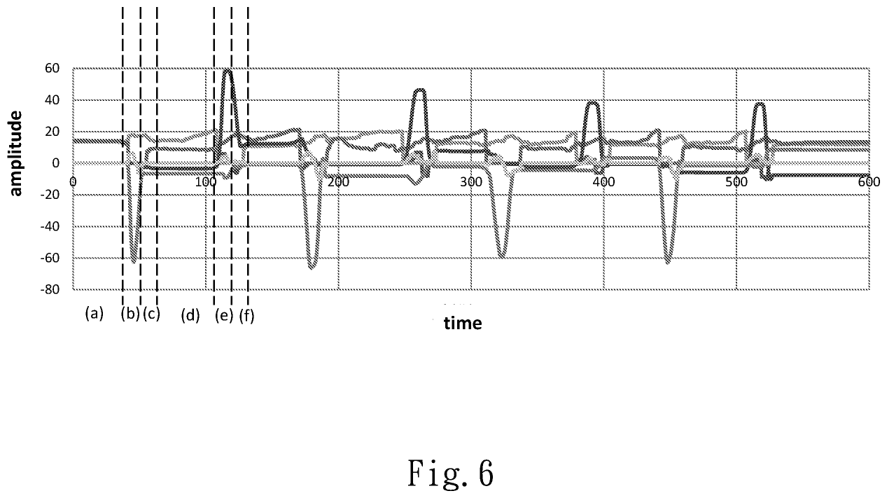

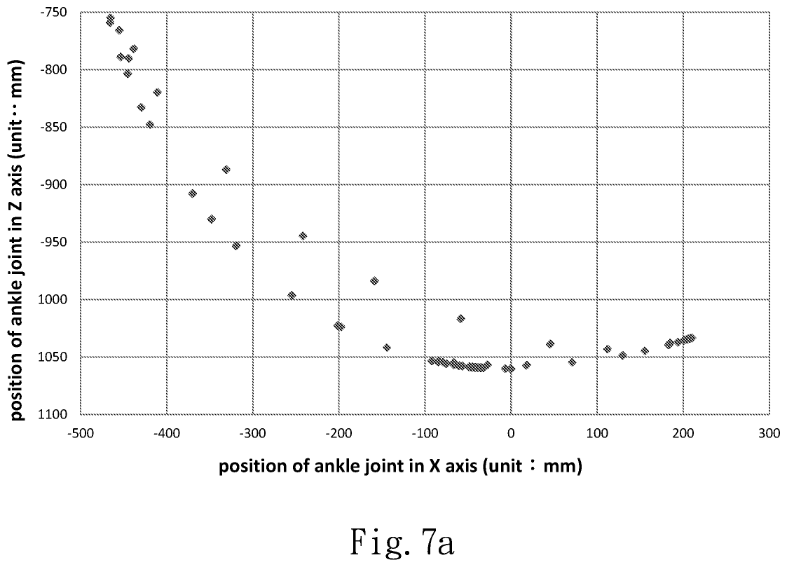

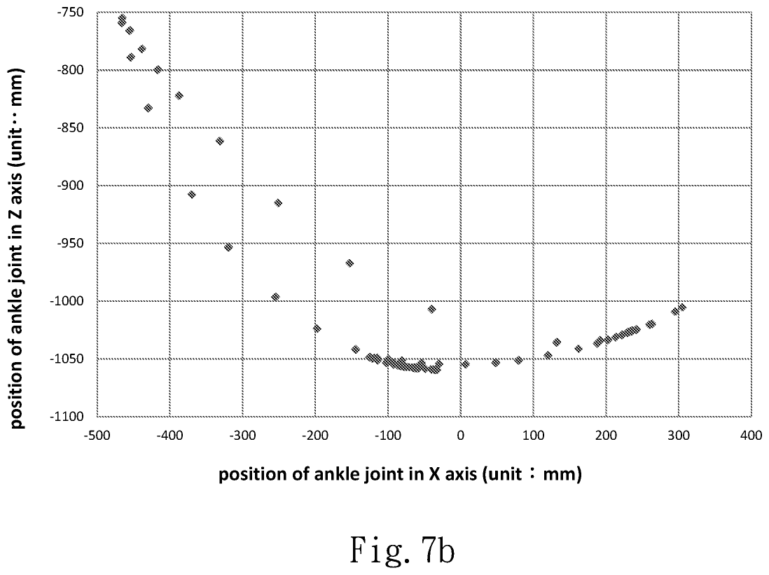

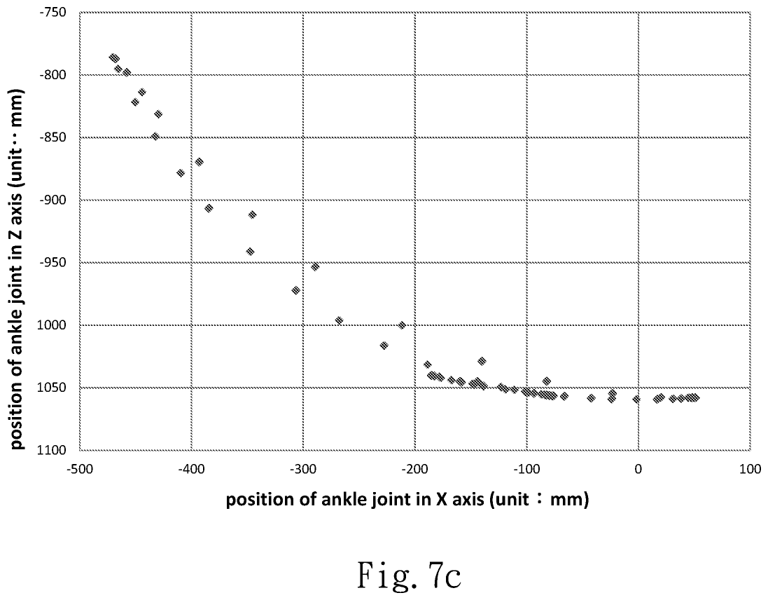

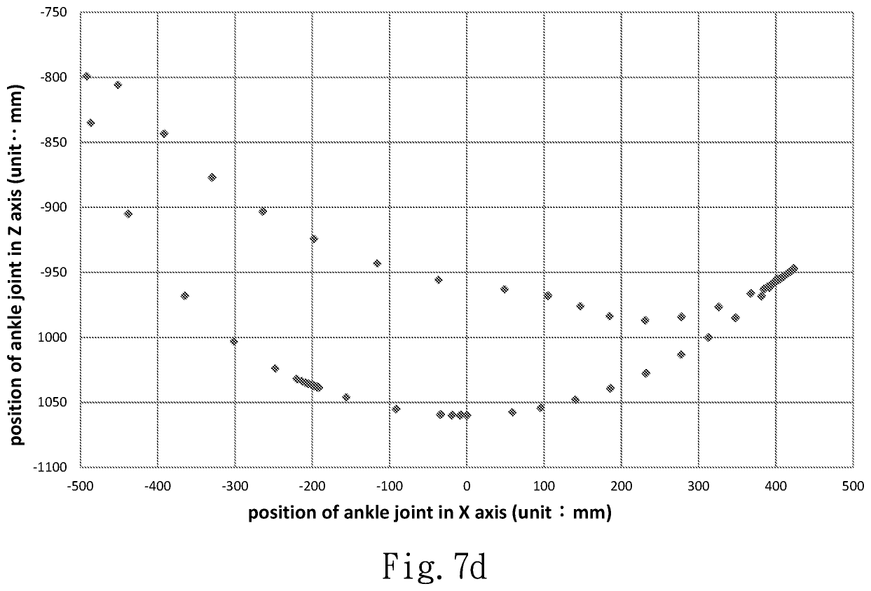

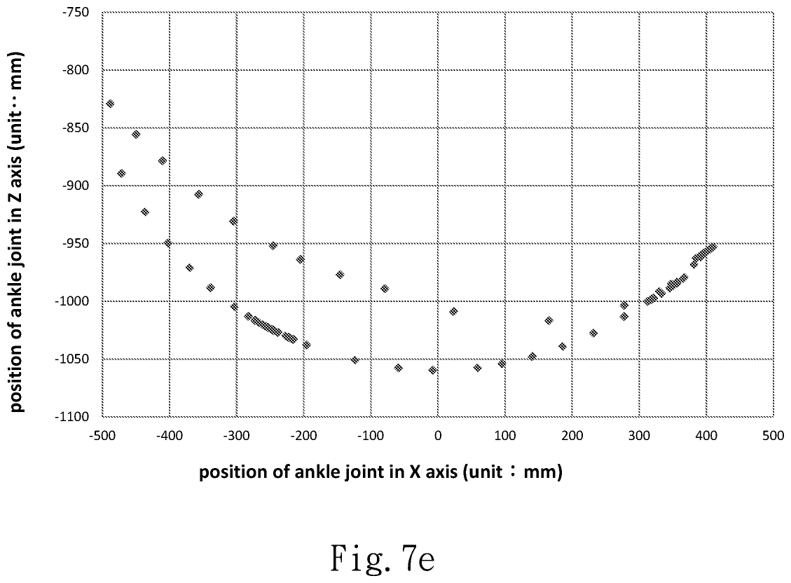

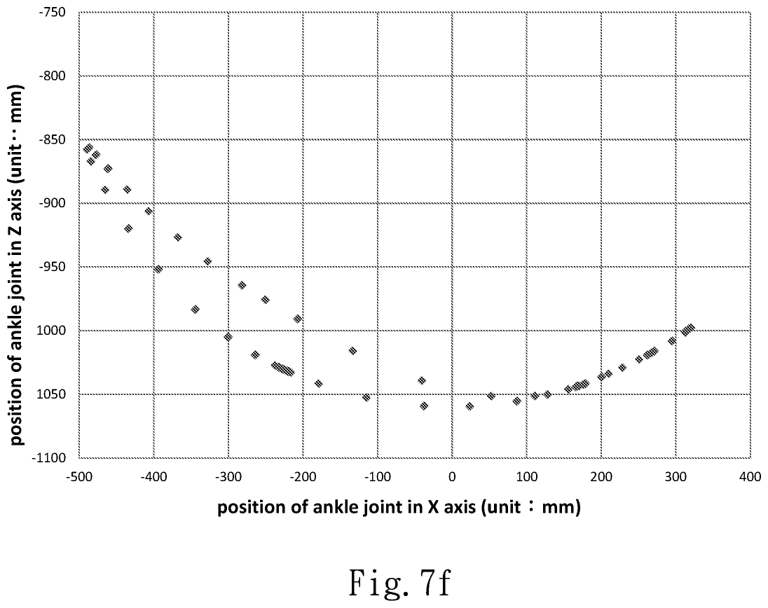

The robot system of the present invention is worn by several users respectively with different heights and weights for simulations that one foot drives the other foot to move, whereby to obtain the similarity between the movement of the driven foot and the movement of the normal foot. The experimental results show that the similarity is over 80%. Therefore, the present invention should be able to provide effective rehabilitation for the patients suffering apoplexy or paralysis in future. Refer to FIG. 5, FIG. 6 and FIGS. 7a-7f. FIG. 5 shows the signals generated by the first rotation device while the hip joints of the users are moving. FIG. 6 shows the signals generated by the second rotation device while the knee joints of the users are moving. FIGS. 7a-7fshow the signals generated by the bottom sustaining device while the knee joints of the users are moving, wherein FIG. 7a is corresponding to Parts (a) in FIG. 5 and FIG. 6; FIG. 7b is corresponding to Parts (b) in FIG. 5 and FIG. 6; FIG. 7c is corresponding to Parts (c) in FIG. 5 and FIG. 6; FIG. 7d is corresponding to Parts (d) in FIG. 5 and FIG. 6; FIG. 7e is corresponding to Parts (e) in FIG. 5 and FIG. 6; FIG. 7f is corresponding to Parts (f) in FIG. 5 and FIG. 6. At the moment that the user raises his foot, the initial value is zero. While the user is moving his feet, the angles of the joints vary with his posture. While the user uses one of his feet to step forward uses the other of his feet to support his body in the rear, the values of two angles of the hip joint have the same sign. In the same condition, the signs of the angles of the knee joints are opposite to each other. In each diagram, Parts (a), (b) and (c) are the motion tracks of the healthy half body, and Parts (d), (e) and (f) are the motion tracks of the paralyzed half body. It is found: in each cycle, the auxiliary force in Parts (e) and (f) for the paralyzed half body is similar to the applied force in Parts (b) and (c) of the healthy half body. Parts (a) and (d) are the motion tracks in standing. The auxiliary torque of the hip joint follows the applied force of the preceding step. The force-applying decision will be updated to vary the value of the auxiliary torque for different postures. While the paralyzed half body of the user insists on a certain posture, the auxiliary torque will be provided persistently. It is found: while the user stands, the angle of the knee joint in standing in Part (d) is near the angle of the knee joint in standing of the former step in Part (a) (the tolerance may be set to be ±10 degrees), whereby the body can be supported.

It has been proved by experiments: even though applied to users with different heights and weights, the present invention can still provide symmetric gaits. Therefore, the present invention can promote rehabilitation effect and apply to rehabilitation centers and clinics, exempting patients from being supported by medical personnel, saving medical resources, and decreasing occupational injuries.

The embodiments have been described in detail to demonstrate the technical thoughts and characteristics of the present invention to enable the persons skilled in the art to understand, make, and use the present invention. However, these embodiments are not intended to limit the scope of the present invention. Any equivalent modification or variation according to the spirit of the present invention is to be also included by the scope of the present invention.

What is claimed is:

1. A walking rehabilitation robot system, which is worn by a user, comprising
two robot feet, wherein one of said robot feet, which is moved firstly, generates a plurality of motion detection signals, and the other of said robot feet learns to move according to said motion detection signals; and
a control device electrically connected with each of said two robot feet, receiving said motion detection signals transmitted by said robot foot moving firstly, working out a first motion track, and working out torques of motors of the other said robot foot according to said first motion track and said motion detection signals, and controlling the other said robot foot to generate a second motion track symmetric to said first motion track.

2. The walking rehabilitation robot system according to claim 1, wherein each of said two robot feet further comprises
a first shaft device electrically connected with said control device and transmitting a first force signal to said control device while moving;
a first rotation device disposed at one end of said first link device, electrically connected with said control device, rotated by actions of said user to generate a first rotation signal and transmit said first rotation signal to said control device; alternatively controlled by said control device to rotate and simultaneously drive said first link device to move;
a second rotation device disposed at the other end of said first shaft device, electrically connected with said control device, rotated by actions of said user to generate a second rotation signal and transmit said second rotation signal to said control device; alternatively controlled by said control device to rotate;
a second link device having said second rotation device disposed at one end thereof, electrically connected with said control device, generating a second force signal and transmitting said second force signal to said control device while moved by rotation of said second rotation device; and
a bottom sustaining device disposed at the other end of said second link device, electrically connected with said control device, sustaining a sole of said user, and transmitting pressure sensation signals to said control device while said user moves said sole to let said control device learn movement of said bottom sustaining device, wherein said control device works out said first motion track according to variations of said first force signal, said second force signal, said first rotation signal, said second rotation signal and said pressure sensation signals.

3. The walking rehabilitation robot system according to claim 2, wherein said first link device includes a first force sensor using resistance variation to detect said first force signal while said first link device is moving.

4. The walking rehabilitation robot system according to claim 3, wherein said second link device includes a second force sensor using resistance variation to detect said second force signal while said second link device is moving.

5. The walking rehabilitation robot system according to claim 2, wherein said bottom sustaining device includes a plurality of pressure sensors detecting applied forces at different positions of said sole of said user to generate said pressure sensation signals, learning movements of said bottom sustaining device from variations of said pressure signals, and working out a center of pressures on said sole.

6. The walking rehabilitation robot system according to claim 1, wherein said control device includes a computer, motor drivers, microcontrollers, and power suppliers.

7. The walking rehabilitation robot system according to claim 1 further comprising a waistband assembly annularly disposed around a waist of said user, carrying said control device, and coupling said two robot feet.

8. The walking rehabilitation robot system according to claim 1, wherein each of said two robot feet includes a gravity compensator electrically connected with said control device and compensating for gravitational influence while said robot foot is moving.

9. The walking rehabilitation robot system according to claim 1, wherein said first motion track and said motion detection signals are used to work out said second motion track corresponding to said first motion track in an Inverse Reinforcement Learning (IRL) method and a Q-learning method.

10. The walking rehabilitation robot system according to claim 1, wherein said control device uses an Inverse Reinforcement Learning (IRL) method to analyze translation, rotation, and acting force in said first motion track and uses a Q-learning method to acquire optimized action inputs corresponding to said translation, said rotation and said acting force, and wherein said control device works out said torques of said motors, which are required by said second motion track, according to said optimized action inputs, and wherein said control device controls said motors to output said torques to make the other said robot foot generate said second motion track symmetric to said first motion track.
